(19) United States
(12) Patent Application Publication
(10) Pub. No.: US 2007/0181125 A1
(43) Pub. Date: Aug. 9, 2007

(54) ventilator safety valve

(76) Inventor: Jan Paul Mulier, Leuven (BE)

Correspondence Address: Jan Paul Mulier, Veldstraat 14, 8200 Brugge, Brugge, 8200, BE

(21) Appl. No.: 10/908,110

(22) Filed: Apr. 28, 2005

(51) Int. Cl. A61M 16/00; A62B 7/00; F16K 31/02

(52) U.S. Cl. 128/204.21; 128/204.18

(57) ABSTRACT

The system and method for preventing volutrauma and barotrauma in patients connected to a ventilator or a manual ventilation system detects elevated airway pressures above a safety pressure and during a time longer than the normally used inspiratory time periods, opens a safety valve to reduce airway pressure below the dangerous values, generates an alarm and closes again the airway system allowing further ventilation. This ventilator safety valve can work repetitively thereby protecting continuously the airway, but at the same time generating a simple method of escape ventilation. Two prototypes of this invention system are build, an electric system driven by computer software written in lab view and a mechanical pneumatic system with a concertina, a spring and an air reservoir under the concertina emptying through a micro opening allowing a time delay before a valve is opened.

### FIELD OF THE INVENTION

The present invention relates to a method that prevents barotrauma and volutrauma when a patient is connected to a ventilator or a manual ventilation system without interfering with the ventilation and without creating a dangerous air leak by being pressure and time sensitive.

### BACKGROUND OF THE INVENTION

It is important to prevent barotrauma and volutrauma. Connecting a patient to a ventilator without starting the ventilator or without allowing the patient to breathe spontaneously could create a totally closed system that is very dangerous. This situation is still possible with the ventilators used today in anaesthesia as with the new build anaesthesia ventilators.

### SUMMARY OF THE INVENTION

The system and method for preventing volutrauma and barotrauma in patients connected to a ventilator or a manual ventilation system detects elevated airway pressures above a safety pressure and during a time longer than the normally used inspiratory time periods, opens a safety valve to reduce airway pressure below the dangerous values, generates an alarm and closes again the airway system allowing further ventilation. This ventilator safety valve can work repetitively thereby protecting continuously the airway, but at the same time generating a simple method of escape ventilation. This ventilator safety valve prevents volutrauma and barotrauma in patients not properly connected to a ventilator or connected to a defective or not working ventilator by being sensitive for elevated airway pressure and time. This allows every possible normal ventilator setting in combination with a full protection against increased airway pressures during a time longer than the maximum inspiratory period

### BRIEF DESCRIPTION OF THE DRAWINGS

In order to better understand the invention and to illustrate it in practice, non-limiting examples of some preferred embodiments will now be described, with reference to the accompanying drawings, in which:

FIG. 1 is a schematic illustration of the device in rest position with closed escape valve without connection to the airway.

FIG. 2 is a schematic illustration of the device with open escape valve when connected to an elevated airway pressure for a long time.

FIG. 3 is a schematic illustration of the device with closed escape valve and open rapid filling 3 when airway pressure drops and device return to normal position.

FIG. 4 is a schematic illustration of the electric device.

### DETAILED DESCRIPTION OF PREFERRED EMBODIMENTS

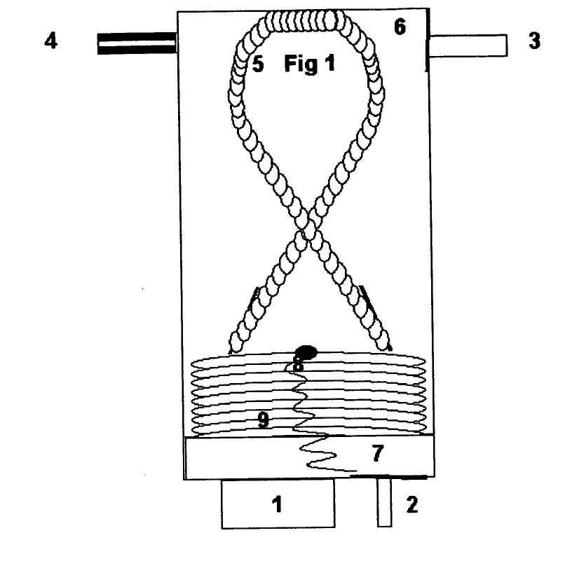

A safety valve device in accordance with a preferred embodiment of the present invention, is shown in FIG. 1 where the device is at rest with escape valve 7 on outlet 2 being closed. The safety valve device is made of a clear Plexiglas cylinder with a concertina bellow inside containing no latex rubber. It is connected to the airway by connection 1. The air of the pressure relief valve is vented to the outside by outlet 2. Rapid filling of the pressure chamber and resetting memory is possible through inlet 3. The air escapes the pressure chamber through a small hole in outlet 4 creating a time period before the chamber is emptied. A spring 5 creates continuously an equal pressure in the concertina independent of concertina position. A one way valve 6 is connected on inlet 3 allowing only filling. A second one way valve 7 is connected on outlet 2. This valve is normally closed because pressure in airway is always equal or higher than ambient air. Valve is opened through attached cord 8. The cord is attached to valve 7 and to the top of concertina 9. The cord is stretched when the concertina moves to the upper position. At that moment it opens valve 7. The concertina 9 divides the room in an upper pressure chamber outside the concertina and a lower pressure chamber under concertina in connection with airway 1.

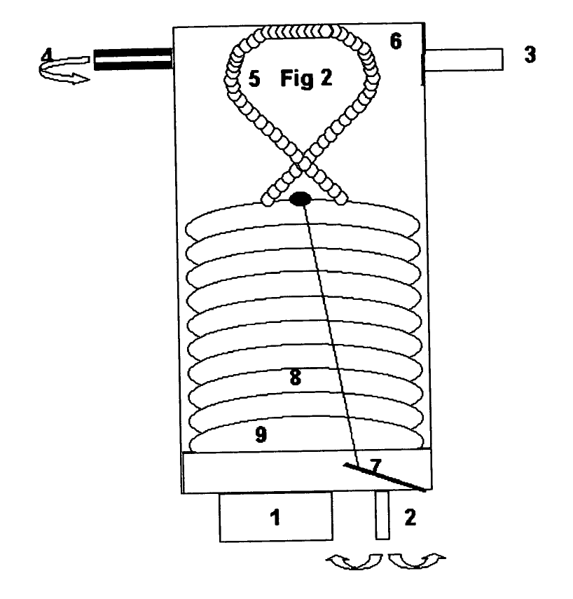

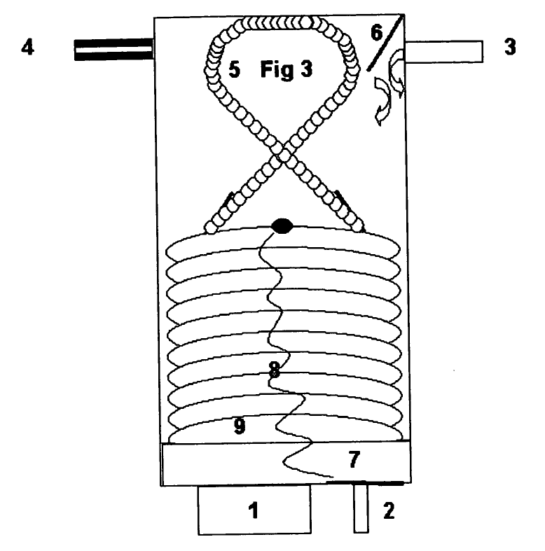

In FIG. 2 the device is connected during a long time to an elevated airway pressure. The concertina 9 is elongated, the cable 8 stretches and the escape valve 7 opens. The air from the airway 1 can escape to outside 2 thereby reducing the airway pressure. When the airway pressure reach zero the spring presses the concertina 9 down as shown in FIG. 3. The inlet valve 6 opens to allow fast entry of outside air through inlet 3 and allow the concertina 9 to return fast to the initial position.

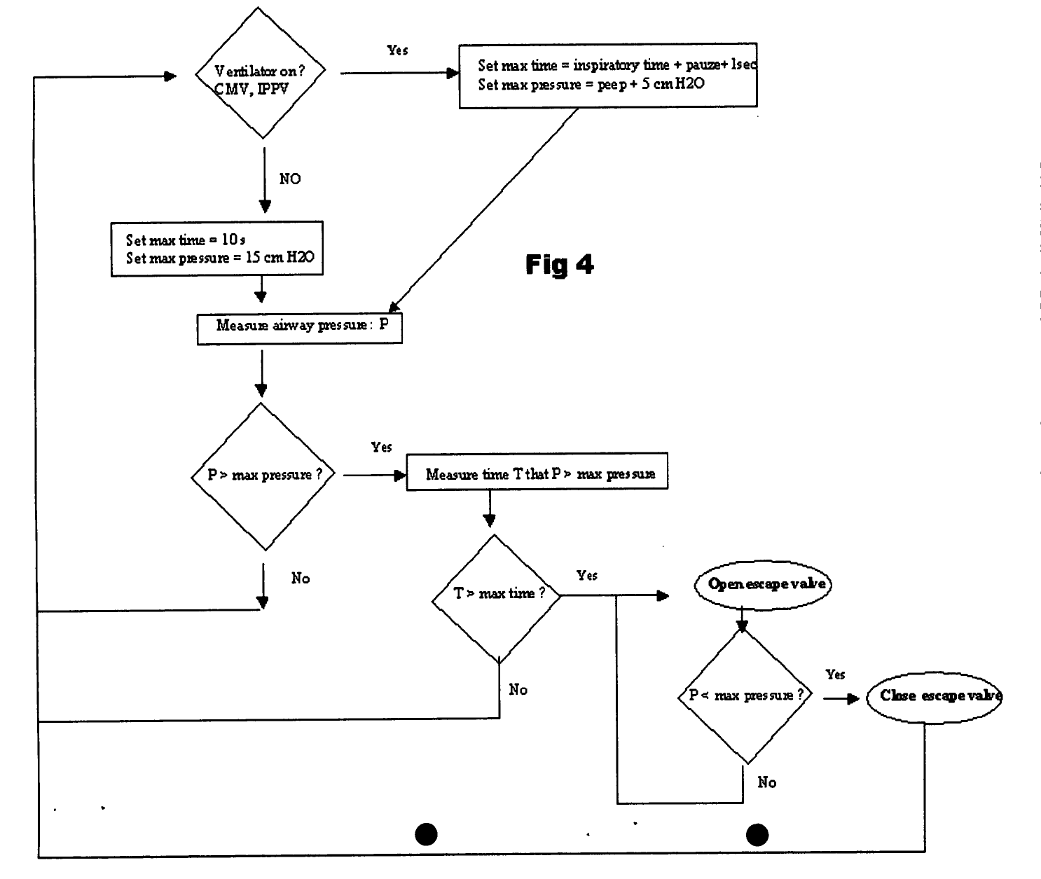

In FIG. 4 a schematic drawing of the electronic analysis system is given. The numbers for time and pressure are given as an illustration of a possible setting. A pressure sensor in the airway pressure is the only required parameter. With an external pressure sensor this system can work alone. Using the internal airway pressure sensor as is available in most modern ventilators this analysis system can build in these ventilators. The computer system requires a clock and has as an output controlling the opening and closing of the safety valve.

What is claimed is:

1. A lung ventilator safety valve apparatus for relieving pressure in a patient airway of a ventilator system, comprising:
a safety valve connected to a patient airway of a ventilator system, said safety valve having a normally closed position and an open position venting pressure from said patient airway;
a pressure sensor means connected to said patient airway for monitoring pressure in said patient airway and for generating a pressure signal indicative of sensed pressure in said patient airway;
a time monitor means connected to pressure sensor means for monitoring time that pressure is elevated in the airway above maximum pressure threshold as measured by pressure sensor
a valve control means connected to said safety valve for controlling operation of said safety valve, and connected to said pressure sensor means for receiving said pressure signal and to said time monitor means for receiving time signal, and having means for setting a maximum airway pressure threshold and a maximum time threshold of elevated inspiratory pressures, a means for comparing said sensed pressure and said monitored time in said patient airway with said maximum airway pressure and time threshold, and means for generating a valve open signal when said sensed pressure and time in said patient airway is equal to or greater than said maximum airway pressure and said maximum time
a valve switching means connected to said valve control means for receiving said valve open signal for switching said safety valve to said open position in response to said valve open signal.

2. The lung ventilator safety valve apparatus of claim 1, wherein said safety valve comprises a pulse width actuated solenoid valve biased to said normally closed position.

3. The lung ventilator safety valve apparatus of claim 1, wherein said means for setting a maximum airway pressure threshold and for setting a maximum time of elevated inspiratory pressures comprises a keyboard.

4. The lung ventilator safety valve apparatus of claim 2, wherein said valve switching means comprises a pulse power source for providing cyclical pulses of power for operating said solenoid valve, and pulse width modulation means for modulating the duration of said pulses for moving said safety valve to said open position.

5. The lung ventilator safety valve apparatus of claim 1, wherein said means for setting a maximum airway pressure threshold and for setting a maximum time of elevated inspiratory pressures comprises a dial.

6. The lung ventilator safety valve apparatus of claim 1, wherein said means generates an alarm when safety valve opens and wherein said means closes again spontaneous the airway system after a specified time allowing further ventilation. This ventilator safety valve can function repetitively thereby protecting continuously the airway and at the same time generating a simple method of escape ventilation.

7. The lung ventilator safety valve apparatus of claim 1, wherein said apparatus functions without electricity power and without electronic regulatory system. This mechanical apparatus for relieving pressure in a patient airway of a ventilator system, comprises:
a mechanically activated safety valve connected to a patient airway of a ventilator system, said safety valve having a normally closed position and an open position venting pressure from said patient airway;
a mechanical pressure sensor means connected to said patient airway for monitoring pressure in said patient airway and for activating safety valve when airway pressure stay elevated above a maximum pressure during a maximum time;
a valve control means connected to said safety valve for controlling operation of said safety valve, and connected to said pressure sensor means for receiving said pressure signal, said valve control means including means for setting a maximum airway pressure threshold and a maximum time threshold of elevated inspiratory pressures, a means for comparing said sensed pressure and said sensed time in said patient airway with said maximum airway pressure and maximum time threshold, and means for generating a valve open signal when said sensed pressure is continuously during said maximum time equal to or greater than said maximum airway pressure.
and a valve switching means connected to said valve control means for receiving said valve open signal and for switching said safety valve to said open position.

8. The lung ventilator safety valve apparatus of claim 7, where maximum pressure and maximum time can be mechanical adapted.
